(19) United States
(12) Patent Application Publication
(10) Pub. No.: US 2008/0015481 A1
(43) Pub. Date: Jan. 17, 2008

(54) HEMOSTATIC BANDAGE AND METHOD OF USE

(76) Inventors: Patrick J. Bergin, Eugene, OR (US); Jeffrey P. Wensel, Eugene, OR (US)

Correspondence Address: ADELI LAW GROUP, A PROFESSIONAL LAW CORPORATION, 1875 CENTURY PARK EAST, SUITE 1360, LOS ANGELES, CA, 90067, US

(21) Appl. No.: 11/671,448

(22) Filed: Feb. 5, 2007

Related U.S. Application Data
(63) Continuation-in-part of application No. 11/245,956, filed on Oct. 7, 2005.
(63) Continuation-in-part of application No. 11/332,784, filed on Jan. 12, 2006.
(63) Continuation-in-part of application No. 11/245,956, filed on Oct. 7, 2005.
(63) Continuation-in-part of application No. 11/332,784, filed on Jan. 12, 2006.
(60) Provisional application No. 60/677,863, filed on May 4, 2005.
(60) Provisional application No. 60/688,510, filed on Jun. 7, 2005.
(60) Provisional application No. 60/693,706, filed on Jun. 24, 2005.
(60) Provisional application No. 60/723,878, filed on Oct. 5, 2005.
(60) Provisional application No. 60/827,055, filed on Sep. 26, 2006.
(60) Provisional application No. 60/863,565, filed on Oct. 30, 2006.

(51) Int. Cl. A61F 13/02

(52) U.S. Cl. 602/46; 602/48; 602/54; 602/56

(57) ABSTRACT

Some embodiments of the invention provide an apparatus for achieving hemostasis in a puncture tract. Such a tract might have been created during a medical procedure, operation or a traumatic injury. In some embodiments, the apparatus includes a bandage for placement within the puncture tract. The apparatus also includes a stem with a distal end. In some embodiments, the bandage couples to the distal end of the stem. In other embodiments, the bandage couples to the entire length of the stem. Along with the bandage, the stem's distal end is at least partially inserted into the puncture tract in some embodiments. In other embodiments, only the bandage is inserted into the puncture tract. The bandage is typically maintained within the puncture tract until hemostasis is achieved. Once hemostasis has been achieved, the operator removes the bandage from the puncture tract.

### CLAIM OF BENEFIT

This application is a continuation-in-part of U.S. patent application Ser. No. 11/245,956, filed on Oct. 7, 2005, and is a continuation-in-part of U.S. patent application Ser. No. 11/332,784, filed on Jan. 12, 2006. Both these non-provisional applications claim the benefit of U.S. Provisional Application 60/693,706, filed on Jun. 24, 2005, and both these non-provisional applications claim the benefit of U.S. Provisional Application 60/688,510, filed on Jun. 7, 2005. The nonprovisional application Ser. Nos. 11/245,956 and 11/332,784 are incorporated herein by reference.

### FIELD OF THE INVENTION

The invention is directed towards a hemostatic bandage and a method of using the same.

### BACKGROUND OF THE INVENTION

Numerous medical diagnostic and therapeutic procedures require access to the internal organs of a living organism. Some of these procedures can be performed without traditional surgical incisions by utilizing catheter-based apparatus to enter blood vessels. Usually, catheter-based apparatus require a needle to be inserted through the skin and directed into a blood vessel. This provides a conduit for extending a metal or polymer guide wire through the needle and into the vasculature. After positioning the guide wire in the conduit, the needle can be removed and replaced with a hollow tube or catheter directed over the guide wire into the blood vessel. The tube or catheter provides access for administration of certain substances and/or for passage of additional equipment that will be used to perform manipulations within the vasculature or within other organ systems accessible through the vasculature.

To prevent bleeding upon completion of a catheter-based intravascular procedure, the catheter must be removed and the puncture site sealed. In the low-pressure environment of the venous system, a small needle puncture is readily sealed by the brief application of pressure to the site and application of a light dressing, such as a bandage. This method is widely utilized after needle stick procedures such as blood drawings.

However, when punctures are created with larger caliber apparatus (such as catheters) in the high-pressure environment of arteries, the puncture created will not readily seal with the application of brief pressure. Prolonged external pressure may be required for fifteen to thirty minutes and may lead to substantial discomfort at the puncture site for the patient and/or a significant failure rate with late bleeding and hematoma formation.

In some instances, puncture wounds may be created by traumatic events occurring outside of a hospital. For example, a traumatic event can be a puncture wound created by bullets, shrapnel, knives, and other penetrating devices. Such wounds also typically do not readily seal with the application of brief pressure.

In the past, several methods have been proposed to address these problems. For instance, for traumatic puncture wounds, external bandages and/or pressure are applied topically. However, this is often inadequate to effectively reduce or eliminate bleeding due to puncture or disruption of blood vessels and organs.

Another approach is to use an apparatus that utilizes a marker to indicate the position to apply pressure with the bandage. Another apparatus uses a pad which, when moistened by fluid from a wound, expands and exerts pressure against the wound. Yet another apparatus utilizes laser energy directed through a balloon tipped catheter into the vascular tract and positioned just outside the outer wall of the blood vessel. The balloon is used to create a covering for the vascular puncture. The laser is used to create a laser “weld” or seal in the adjacent tissue.

Another apparatus uses both a balloon tipped catheter and an absorbable plug. The plug is used to occlude the vascular access tract and provide hemostasis. The balloon tipped catheter serves as a positioning anchor for antegrade insertion of the vascular plug and must be removed from the patient after plug deployment.

Still another apparatus uses a balloon tipped catheter arranged so as to pass into the vascular lumen by means of the extant access sheath. After this procedure, it is withdrawn from the intraluminal side of the blood vessel puncture to provide temporary hemostasis. A pro-coagulant slurry is then injected into the vascular access tract to promote coagulation. During this time, the balloon tipped catheter remains inflated. After a suitable period of time necessary to promote blood coagulation, the balloon tipped catheter is deflated and withdrawn from the access tract.

Each of these approaches has its own unique set of shortcomings. The prior apparatus lack both a mechanism for precise positioning of a pressure-generating component against a puncture tract and a structure designed to optimize the pressure that is to be applied to such a site. Therefore, there is a need in the art for an apparatus that hemostatically closes a vascular puncture site without leaving a hematoma within the puncture tract, while minimizing patient discomfort. Ideally, such an apparatus would quickly, painlessly and reliably achieve hemostasis upon withdrawal of the vascular catheters. Consequently, such an apparatus would reduce patient discomfort, staff time and the unfavorable failure rate associated with vascular hemostasis and the risk of hemotoma formation.

### SUMMARY OF THE INVENTION

Some embodiments of the invention provide an apparatus for achieving hemostasis in a puncture tract. Such a tract might have been created during a medical procedure or operation. Alternatively, the tract might be a result of a traumatic injury (e.g., injury that occurred outside of a hospital) that created a traumatic wound, such as a bullet wound, shrapnel or knife puncture. The puncture typically extends from the epidermis to the vasculature and/or internal organs in a living organism.

In some embodiments, the apparatus includes a bandage for placement within the puncture tract. The apparatus also includes a stem with a distal end. In some embodiments, the bandage couples to the distal end of the stem. In other embodiments, the bandage couples to the entire length of the stem. In some embodiments, the bandage is wire-guided into the puncture tract. Along with the bandage, the stem's distal end is at least partially inserted into the puncture tract in some embodiments. In other embodiments, only the bandage is inserted into the puncture tract.

The bandage is typically maintained within the puncture tract until hemostasis is achieved. The apparatus in some embodiments includes a handle that allows an operator to apply pressure to maintain the bandage in the puncture tract until hemostasis is achieved. While in the puncture tract, the bandage occludes the puncture tract. Accordingly, the bandage is also referred to below as a “plug” as it blocks the puncture tract. In some embodiments, the stem further occludes the puncture tract, while the stem and/or handle occlude the opening of the puncture tract. Once hemostasis has been achieved, the operator removes the bandage from the puncture tract.

In some embodiments, the bandage is composed of polymeric foam. The polymeric foam is hydrophilic polyurethane foam in some embodiments. The bandage can have many shapes, but typically has a shape that facilitates its insertion into the puncture tract. For instance, in some embodiments, the bandage has a tapered tip.

Also, in some embodiments, the bandage includes or is coated with one or more materials that are designed to promote coagulation and thereby achieve hemostasis. For example, in some embodiments, the bandage includes, is coated with, or is entirely composed of Chitosan. Chitosan may be incorporated on and into the foam bandage by means of dipping the bandage into a Chitosan solution. In some embodiments, the foam bandage is lyophilized after dipping it into the Chitosan solution.

### BRIEF DESCRIPTION OF THE DRAWINGS

The novel features of the invention are set forth in the appended claims. However, for purpose of explanation, several embodiments of the invention are set forth in the following figures.

FIG. 1 illustrates a side elevation view showing in cross section, a hemostasis sheath placed over a guide wire within a blood vessel through the skin of a living being.

FIG. 2 illustrates a side elevation view showing in cross section, a guide wire in place with the hemostasis sheath removed.

FIG. 3 illustrates a side elevation view showing in cross section, an occlusive bandage being passed over the guidewire and into the puncture tract.

FIG. 4 illustrates a side elevation view showing in cross section, the occlusive bandage in place with the guide wire removed and the hemostatic bandage secured within the puncture tract.

FIG. 5 illustrates a side elevation view of the component parts of the occlusive bandage.

FIG. 6 illustrates an oblique three dimensional exploded view of the component parts of the occlusive bandage.

FIG. 7 illustrates side perspective view of a hemostatic apparatus according to some embodiments of the invention.

FIG. 8 illustrates a bottom perspective view of the hemostatic apparatus of some embodiments.

FIG. 9 illustrates a top perspective view of the hemostatic apparatus of some embodiments.

FIG. 10 illustrates an exploded and compacted view of the hemostatic apparatus of some embodiments.

FIG. 11 illustrates a cross sectional view of the cover pad and bandage of the hemostatic apparatus of some embodiments.

FIG. 12 illustrates a cross sectional view of the layers of a patient's skin of some embodiments.

FIG. 13 illustrates a cross sectional view of a hemostatic apparatus of some embodiments inserted into the epidermis skin layer.

FIG. 14 illustrates a cross sectional view of a hemostatic apparatus of some embodiments inserted into the dermis skin layer.

FIG. 15 illustrates a cross sectional view of a hemostatic apparatus of some embodiments inserted into the subcutaneous tissue skin layer.

FIG. 16 illustrates a cross sectional view of a bandage of some embodiments with a mild tapered tip.

FIG. 17 illustrates a cross sectional view of a bandage of some embodiments with a sharp tapered tip.

FIG. 18 illustrates a cross sectional view of the hemostatic apparatus of some embodiments.

FIG. 19 illustrate side perspective view of a hemostatic apparatus according to some embodiments of the invention.

FIG. 20 illustrates a top perspective view of the hemostatic apparatus of some embodiments.

### DETAILED DESCRIPTION OF THE INVENTION

In the following description, numerous details are set forth to provide a better understanding of the various embodiments of the invention. However, one of reasonable skill in the art will realize that the invention may be practiced without the use of the specific details presented herein. In some instances of describing the invention, well-known structures and apparatus may be shown in block diagram form to avoid obscuring the description of the invention with unnecessary detail. Therefore, the examples provided herein for clarification and understanding should not be read into and thereby limit the language of the claims.

Some embodiments of the invention provide an apparatus for achieving hemostasis in a puncture tract. Such a tract might have been created during a medical procedure or operation. Alternatively, the tract might be a result of a traumatic injury (e.g., injury that occurred outside of a hospital) that created a traumatic wound, such as a bullet wound, shrapnel or knife puncture. The puncture typically extends from the epidermis to the vasculature and/or internal organs in a living organism.

In some embodiments, the apparatus includes a bandage for placement within the puncture tract. The apparatus also includes a stem with a distal end. In some embodiments, the bandage couples to the distal end of the stem. In other embodiments, the bandage couples to the entire length of the stem. In some embodiments, the bandage is wire-guided into the puncture tract. Along with the bandage, the stem's distal end is at least partially inserted into the puncture tract in some embodiments. In other embodiments, only the bandage is inserted into the puncture tract.

The bandage is typically maintained within the puncture tract until hemostasis is achieved. The apparatus in some embodiments includes a handle that allows an operator to apply pressure to maintain the bandage in the puncture tract until hemostasis is achieved. While in the puncture tract, the bandage occludes the puncture tract. Accordingly, the bandage is also referred to below as a “plug” as it blocks the puncture tract. In some embodiments, the stem further occludes the puncture tract, while the stem and/or handle occlude the opening of the puncture tract. Once hemostasis has been achieved, the operator removes the bandage from the puncture tract.

In some embodiments, the bandage is composed of polymeric foam. The polymeric foam is hydrophilic polyurethane foam in some embodiments. The bandage can have many shapes, but typically has a shape that facilitates its insertion into the puncture tract. For instance, in some embodiments, the bandage has a tapered tip.

Also, in some embodiments, the bandage includes or is coated with one or more materials that are designed to promote coagulation and thereby achieve hemostasis. For example, in some embodiments, the bandage includes, is coated with, or is entirely composed of Chitosan. Chitosan may be incorporated on and into the foam bandage by means of dipping the bandage into a Chitosan solution. In some embodiments, the foam bandage is lyophilized after dipping it into the Chitosan solution.

Several more detailed embodiments of the invention are discussed in Sections III and IV. These embodiments provide a hemostatic bandage and a hemostatic wire-guided bandage delivery system. Section V elaborates on how different embodiments of the invention deliver their hemostatic bandage to different depths within or through the skin. Section VI then elaborates on the structure and composition of the hemostatic bandage of some embodiments. However, before discussing these embodiments, it is helpful to understand relevant terminology and some environments in which the hemostatic bandage and its associated delivery system are used. Therefore, Section I presents relevant terminology, while Section II provides an overview of intravascular procedures, which are one type of procedure in which some embodiments can be used.

### I. TERMS AND TERMINOLOGY

An opening in the skin is called a percutaneous opening because it passes through the skin. The skin can be described in terms of the epidermis skin layer, dermis skin layer, and subcutaneous tissue skin layer. The hole from the percutaneous opening to the blood vessel is the puncture tract or access tract. The terms puncture tract and access tract are used interchangeably throughout the specification. The puncture tract (i.e., access tract) can be caused by a traumatic injury or wound, which creates an opening to the body cavity, organs, or blood vessels. Alternatively, such a tract can be created during a medical procedure or operation.

The opening in the blood vessel wall is a vascular puncture or vascular opening. The open space within the blood vessel is called the vascular lumen. As used in the following discussion, a “lumen” is an opening, such as the cavity of a tubular organ or the bore of a tube (as of a hollow needle or catheter). The term “bandage” is used generically to refer to an apparatus that assists in achieving hemostasis of a wound.

### II. AN EXEMPLARY INTRAVASCULAR PROCEDURE

Some embodiments of the invention have particular utility when utilized in conjunction with intravascular procedures. Today, intravascular procedures are performed by many physicians, such as radiologists and cardiologists. Examples of intravascular procedures include angiography, angioplasty, vascular stenting and stent graft placement, arterial thrombectomy, arterial embolization, intra-arterial drug administration, etc. These procedures normally involve the insertion of a hollow needle (e.g., an 18 gauge thin walled needle) through the skin. The needle is advanced through the body tissue overlying a blood vessel and continued through the proximal side of the vascular wall until the distal tip of the needle enters the vascular lumen. A brisk return of blood through the needle hub signals entry of the needle into the vascular lumen.

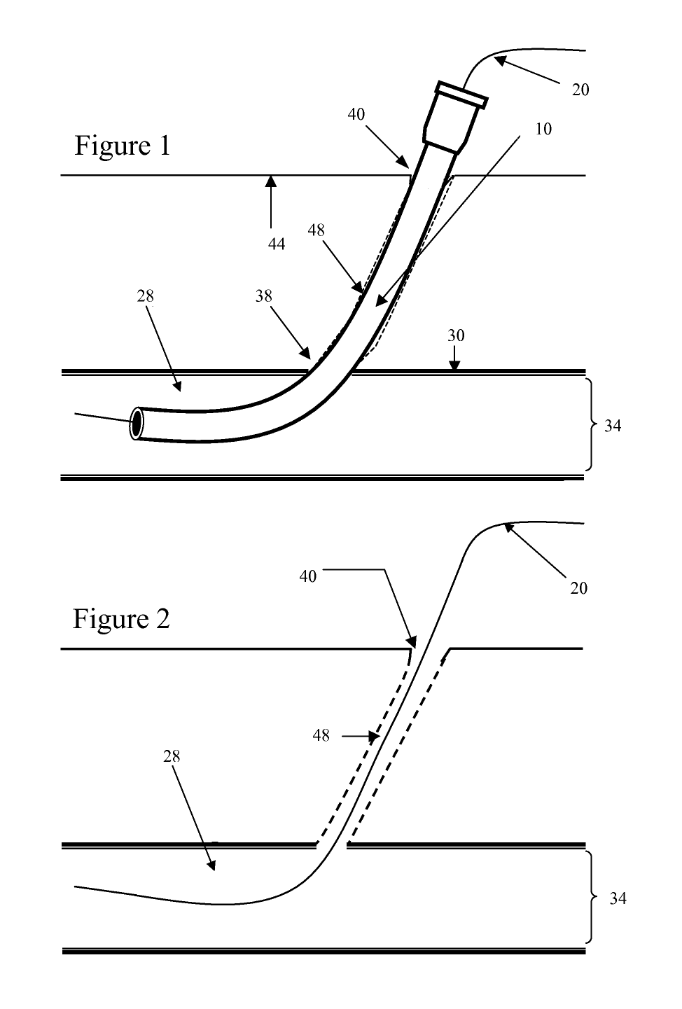

FIGS. 1 and 2 illustrate an exemplary intravascular procedure that commonly uses an access sheath 10 placed in the access tract 48 to facilitate entry into the vascular lumen 34 by diagnostic and therapeutic tools. FIG. 1 illustrates the hemostasis access sheath 10 threaded onto a guide wire 20 and placed within the access tract 48.

To install the access sheath 10, the operator first creates an access path to the blood vessel 28 by cutting a percutaneous opening 40 in the epidermal skin layer 44 at a point that is favorable to accessing the blood vessel 28. A needle (or other cutting tool) is typically advanced through a percutaneous opening 40, an epidermal skin layer 44, a dermal skin layer, a subcutaneous tissue skin layer and a vascular wall 30. It continues through the vascular wall 30 (creating a vascular puncture 38) and into a vascular lumen 34 of a blood vessel 28. This creates the access tract 48.

After creating the access tract 48, the operator typically threads a guidewire 20 longitudinally through the needle. After positioning the guidewire 20 within the access tract 48, the needle may be removed while maintaining the guidewire 20 in position. Normally, an access sheath 10 is later placed within the access tract 48 to prevent the tract 48 from closing during the procedure. The access sheath 10 is typically threaded onto the guidewire 20 and inserted into the access tract 48, using the guidewire 20 to precisely position the sheath 10 into place. When positioned at its final location, one end of the sheath 10 is within the vascular lumen 34 while the opposing end is outside of the organism. Once the access sheath 10 is in place, other apparatus and/or materials can pass through the access sheath 10 and advance into the blood vessel 28 to the area of interest within the body, in order to perform the intravascular procedure.

Upon completion of the intravascular procedure, the catheters and other apparatus used in the procedure are removed from the blood vessel 28 and/or the puncture tract 48. This is generally followed by the removal of the sheath 10 over the guide wire 20, leaving the guide wire 20 in place within the access tract 48 and leaving the access tract 48 open. FIG. 2 presents a longitudinal cross-sectional side view of the access tract 48 with the guidewire 20 in place after the removal of the access sheath 10.

The removal of tools from the access tract 48 causes the access tract 48 to gradually close upon any objects remaining within the tract 48. If hemostasis is not quickly attained, vigorous bleeding can occur. Therefore, the access tract 48 must be sealed as quickly and as efficiently as possible. One method of doing so uses a hemostatic wire guided bandage delivery and placement apparatus of some embodiments of the invention.

### III. HEMOSTATIC BANDAGE AND WIRE-GUIDED DELIVERY SYSTEM FOR DELIVERING THE HEMOSTATIC BANDAGE IN A PUNCTURE TRACT

Some embodiments provide a hemostatic bandage for achieving hemostasis in a puncture tract that is created during a medical procedure. Some embodiments also include a wire-guided delivery mechanism for delivering the bandage into the puncture tract and for maintaining the bandage in the puncture tract until hemostasis is achieved. In some embodiments, the mechanism not only positions the bandage, but also occludes the opening of the puncture tract. Some of the embodiments described below of a hemostatic wire guided bandage delivery and placement apparatus achieve hemostasis at or near a vascular puncture site in a human patient after an intravascular procedure. However, the apparatus' construction and use also have applicability in other settings, e.g., for non-intravascular procedure or for non-human patients (i.e., other living organisms).

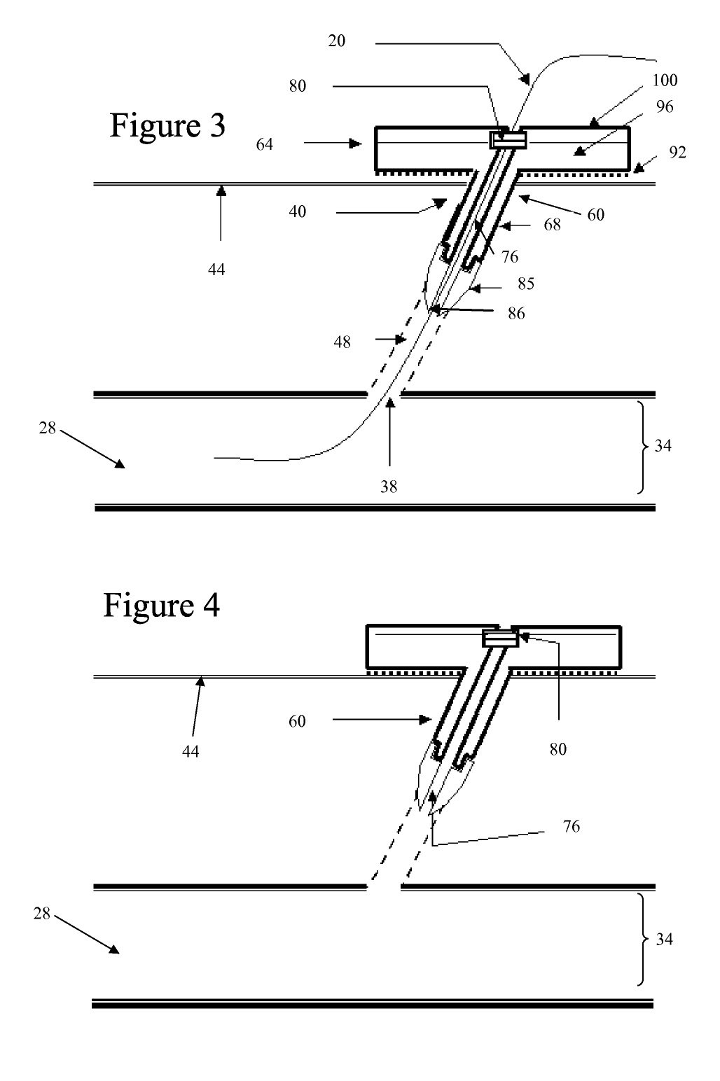

FIGS. 3 through 5 illustrate a hemostatic apparatus 60 of some embodiments of the invention. This apparatus 60 includes a hemostatic bandage 85 and its associated wire guided delivery apparatus. As shown in FIG. 3, the apparatus 60 includes (1) a cover pad 64, (2) a stem 68 affixed to the cover pad 64 and extending at an angle downwards from the bottom side of the cover pad 64, (3) a bandage 85 to the distal end of the stem 68, and (4) a central lumen 76 defined from the top of the cover pad downwards through the center of the stem 68 and through the center of the bandage 85. As shown in this figure, the cover pad 64 includes a hemostatic valve 80.

As shown in FIG. 3, the apparatus 60 positions the bandage 85 within the puncture tract 48 (e.g., subcutaneously within the tract) to provide hemostasis within a puncture tract 48. In use, the cover pad 64 of the apparatus 60 covers and/or occludes the access tract 48 percutaneously. The cover pad's hemostatic valve 80 prevents blood from flowing back through the central lumen 76 and out of the patient, while allowing for the passage of the guidewire 20 through the central lumen 76.

The stem 68 positions the bandage 85 within the access tract 48 to achieve hemostasis. As mentioned above, the stem 68 can extend downwards at an angle from the bottom side of the cover pad 64. This angle corresponds to the angle of the puncture tract 48. In some embodiments, the angle at which the stem 68 extends downwards from the cover pad 64 is adjustable to match angle of the puncture tract 48.

While FIG. 3 presents the guidewire 20 threaded through the apparatus 60, FIG. 4 presents the apparatus 60 after the guidewire 20 has been removed. The guidewire 20 is used to properly guide the bandage 85 as the apparatus 60 is advanced into the access tract 48. After the apparatus 60 is in place, the guidewire 20 may be removed, as shown in FIG. 4. Its removal from the access tract 48 causes the access tract 48 to gradually close further.

The cover pad 64, stem 68 and bandage 85 of the apparatus 60 are discussed in detail in Section A, immediately below. This discussion is followed in Section B by a description of how the apparatus 60 is used in some embodiments to place a hemostasis bandage 85 within a puncture tract 48.

A. The Components of a Bandage Delivery and Placement Apparatus

1. The Cover Pad

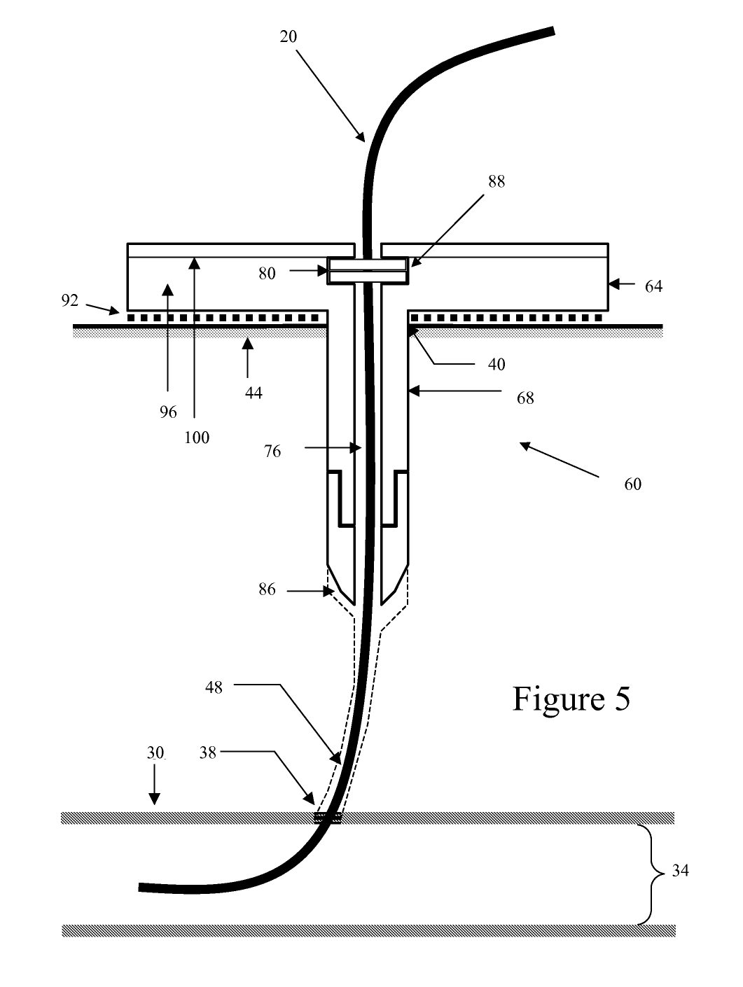

In some embodiments, the cover pad 64 provides a mechanism (1) to act as a handle for holding and maneuvering the apparatus 60, (2) to push or pull the stem 68 into or out of the access tract 48, (3) to occlude the percutaneous opening 40, and (4) affix the apparatus 60 to the epidermal layer 44 during recovery. FIG. 5 presents a more detailed view of the apparatus 60. As shown in this figure, the apparatus 60 in some embodiments includes a multi-layered cover pad 64. The layers include a first adhesive layer 92, a second central layer 96 and a third surface layer 100. The cover pad in some embodiments includes a fourth layer (not shown in FIG. 5) that covers the first adhesive layer 92 as further described below. Although FIG. 5 shows a particular multi-layered cover pad, a person skilled in the art will realize that the cover pad 64 in other embodiments might be constructed differently (e.g., with more or less layers).

As mentioned above, the first layer 92 of the cover pad 64 in some embodiments is an adhesive layer that is applied to the bottom side of the second central layer 96. The first adhesive layer 92 is covered by a fourth layer (not shown) when the bandage has not been deployed. The fourth layer protects the adhesive layer 92 from degradation before the bandage 85 has been deployed. As further described below, the fourth layer is removed from the first layer 92 when the bandage is being deployed, in order to enable the first layer 92 to affix the apparatus 60 to the patient's skin during the procedure.

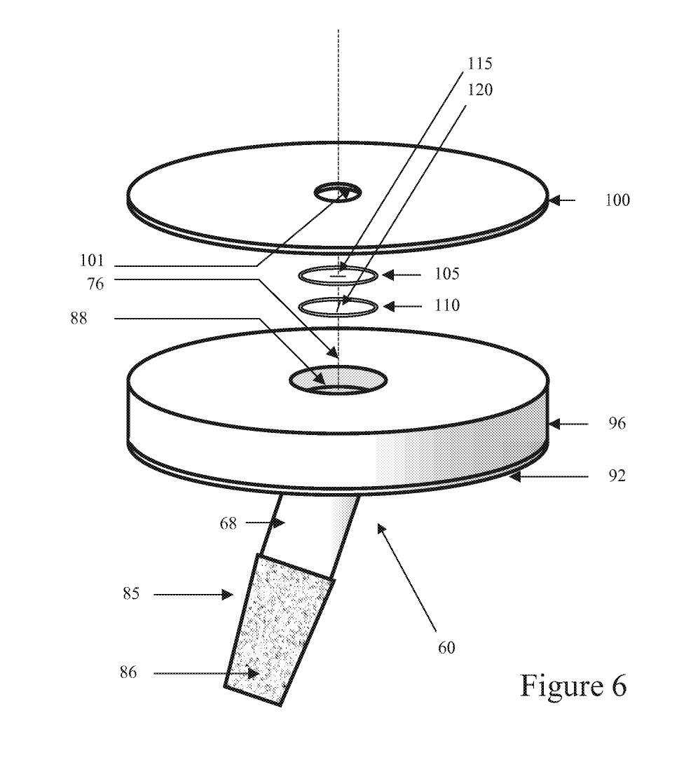

The second layer 96 has a second layer lumen 88 defined about the central lumen 76, which passes through the second layer 96. The hemostatic valve 80 is seated in the second layer lumen 88, which is larger than, and concentric to, the central lumen 76 and is shaped to receive the valve 80. With the valve 80 seated in the second layer lumen 88, the third layer 100 covers the second layer 96 (including the valve 80) to immobilize the valve 80 within the second layer lumen 88. As shown in FIG. 6, the third layer 100 contains a third layer lumen 101 that is concentric to the central lumen 76 and shaped to cooperate with and receive a portion of the hemostatic valve 80 seated in the second lumen 88.

FIG. 6 illustrates an exploded view of the cover pad 64 of some embodiments of the invention. As shown in this figure, the second layer lumen 88 of the second central layer 96 is larger than the third lumen 101 of the third layer 100. This figure also shows that in some embodiments the hemostatic valve 80 is formed by two circular pads 105 and 110.

The circular pads 105 and 110 are formed from a soft rubber material in some embodiments, while they might be formed by other materials in other embodiments. The pads have two slits 115 and 120 at a 90° angle with each other. These two slits allow the guide wire 20 to pass through the central lumen 76. However, the 90° arrangement of the slits plus the composition of the pads 105 and 110 limit the back flow of blood from the central lumen 76. Although the valve 80 is formed by two pads 105 and 110 in some embodiments, one of ordinary skill will realize that the valve 80 is formed differently (e.g., with different number of pads, different composition for the pads, different shaped pads, different type of valve, etc.) in other embodiments.

2. The Stem

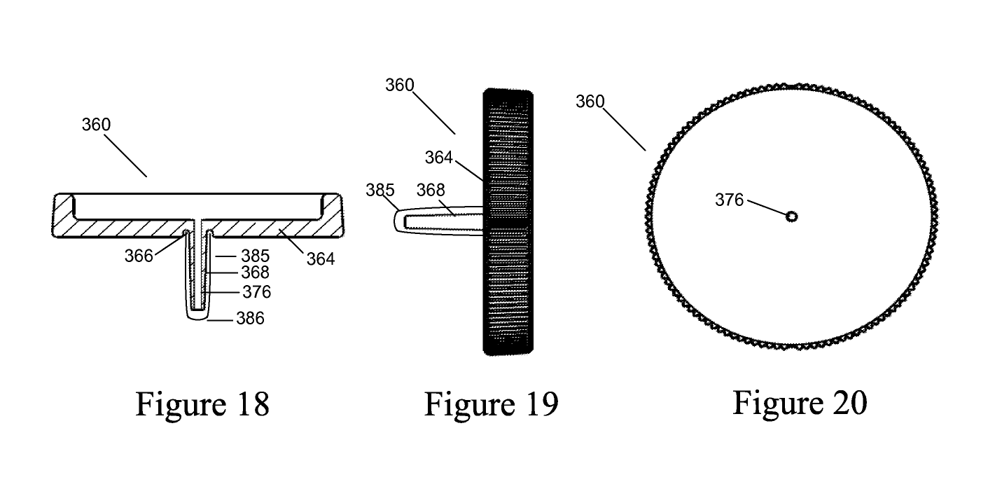

As mentioned above, the stem 68 allows the bandage 85 affixed to the stem 68 to be placed in the puncture tract 48. In some embodiments, the stem 68 is cylindrical and includes a proximal end and an opposing distal end. In other embodiments, the stem 68 is conical, approximately conical/cylinder, or any other shape. The proximal end is affixed to the cover pad 64. In some embodiments, the distal end cooperates with the bandage 85 to place the bandage 85 within the access tract 48. In different versions of the apparatus 60, the stem 68 may have different lengths, in order to position the bandage 85 at different depths within the access tract 48 based upon patient's circumstances. In some embodiments, the stem may be entirely covered by the bandage, as further described below in Section VI by reference to FIGS. 18, 19, and 20. Alternatively, in some embodiments, the stem 68 is a telescopic stem that is capable of achieving different lengths by extending and contracting (i.e., the length of the stem 68 is extended of shortened by telescoping the stem 68). In other embodiments, the stem 68 may be sectioned and joined together, one section at a time, to create an appropriate length for each individual need (i.e., the length of the stem 68 allows adjustments by being sectioned and joined together, one section at a time).

The stem 68 allows the bandage 85 to be placed within the access tract 48 without causing the bandage 85 to flatten near the epidermal layer 44. In so doing, the bandage 85 is placed closer to the vascular puncture 38 in the vascular wall 30 or deeper within a traumatic puncture wound and the chance of hematoma or other undesirable effects is reduced. Section V below elaborates on how different embodiments of the invention deliver a hemostatic bandage (e.g., bandage 85) to different depths within or through the skin.

3. The Bandage

As mentioned above, the bandage 85 is located at the distal end of the stem 68 in some embodiments. The bandage 85 serves to occlude the access tract 48 and provide hemostasis within the access tract 48 without undesirable side effects. In some embodiments, the bandage 85 is a plug that contains a central lumen 76 designed to accept the guidewire 20 and is a component of the delivery apparatus 60. As shown in FIGS. 3-6, the plug 85 has a tapered tip 86 in some embodiments to facilitate entry into the puncture tract. A tapered tip (e.g., tip 86) can simplify the bandage's entry into the puncture tract 48 and thereby allows the bandage 85 to be inserted into the puncture tract 148 without the use of a wire to guide it. The tapered tip 86 can have an angle that is sharp or mild as described in Section VI below.

The depth at which the plug 85 is positioned in the access tract 48 will be approximately the length of the stem 68. In some embodiments, the circumference of the plug 68 is approximately the diameter of the access tract 48. The bandage 85 and its delivery mechanism can be customized for different operating environments by varying their attributes (e.g., varying the length of the stem 68, the dimensions of the bandage 85, etc.).

Some of the embodiments of the bandage 85 may be made from, or coated with, one or more pro-coagulating materials or agents. Pro-coagulating (i.e., coagulating) materials and agents facilitate coagulation and hemostasis. One such coagulation material is Chitosan. By including one or more coagulating materials or agents on or within the bandage 85, hemostasis can be achieved earlier than otherwise achievable. By varying the composition of the bandage 85, the hemostasis rate may be controlled or varied. In this manner, the hemostasis rate may be controlled to fit the needs of each individual circumstance.

Section VI below will further elaborate on the structure and composition of the hemostatic bandage of some embodiments.

B. Method of Use

As discussed previously, removing the access sheath 10 from the access tract 48 at the completion of an intravascular procedure causes the access tract 48 to naturally collapse onto the guidewire 20. Therefore, the apparatus 60 should be inserted into the access tract 48 before the tissue collapses onto the access tract 48. To be most effective, the operator should be able to insert the apparatus 60 quickly, easily and efficiently.

At the conclusion of an intravascular medical procedure, most of the instrumentation used in the procedure is typically removed from the blood vessel and the access tract. For instance, all the instrumentation except the access sheath 10 might be removed from the blood vessel and the access tract 48. Next, a guidewire 20 is re-inserted into the access tract 48 (e.g., re-inserted through the access sheath) and the sheath 10 is then removed.

To insert the apparatus 60, the apparatus is first threaded onto the guidewire 20 by inserting the side of the guidewire 20 that is out of the patient through the hole in the tip 86 of the plug 85, through the central lumen 76, through the slits 115 and 120 of the valve 80, and out of the cover pad 64. The cover for the adhesive layer 92 of the pad 64 is removed to reveal the adhesive layer 92. Next, the apparatus 60 is advanced into the access tract 48 until the bandage 85 is properly placed at the appropriate depth within the puncture tract and the adhesive layer 92 comes in contact with the epidermis 44. Different embodiments deliver the hemostatic bandage 85 to different depths within the puncture tract 48. Section V elaborates on how different embodiments of the invention deliver the hemostatic bandage to different depths within or through the skin.

With the adhesive layer 92 exposed, the cover pad 64 can firmly adhere to the epidermal layer 44 to prevent the bandage 85 from moving within the access tract 48. With the apparatus 60 properly positioned, the guidewire 20 can be removed, as shown in FIG. 4.

With the apparatus 60 in place, the hemostatic valve 80 prevents back bleeding through the central lumen 76. The bandage 85 acts to seal the remaining portion of the access tract. By placing the apparatus 60 within the access tract 48, the bandage 85 and the cover pad 64 both obstruct the flow of blood from the vascular puncture 38.

In some embodiments, the bandage 85 is coated with, contains, or is completely composed of Chitosan or other pro-coagulant (i.e., coagulant) material (i.e. coagulating agents). The use of coagulating agents in the bandage 85 further impedes the blood flow. Section VI further elaborates on the structure and composition of the bandage of some embodiments. Next, the removal of the guidewire 20 causes the access tract 48 to collapse. Also, the tissue exerts force on the tapered tip 86 of the bandage 85 to close the hole at this tip 86. The insertion of the bandage, the use of the coagulating agents, and the collapse of the tissue restrict the flow of blood from the blood vessel 28 and thereby quickly and efficiently result in hemostasis. To achieve hemostasis, a physician might also exert minimal pressure on the cover pad 64 in some cases for a small duration of time (e.g., thirty to sixty seconds). Also, the bandage 85 has to remain in the patient for a suitable amount of time to secure hemostasis. This amount can be as little as 2 to 60 minutes in some cases.

After a suitable period to allow for recovery and healing, the bandage 85 is removed from the living organism by pulling the cover pad away from the patient. After the removal of apparatus 60, a light topical dressing is then applied to the wound.

### IV. HEMOSTATIC BANDAGE WITH LUBRICIOUS SHEATH

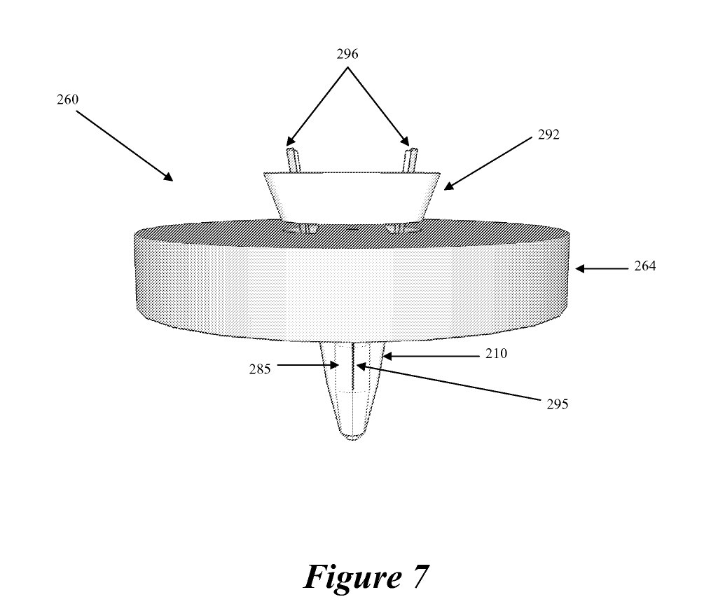

FIGS. 7-11 illustrate another example of a hemostatic apparatus 260 of some embodiments of the invention. The hemostatic apparatus 260 has many of the same features as the hemostatic apparatus 60 illustrated in FIGS. 3-5. For instance, as illustrated in FIGS. 7-10, the apparatus 260 includes (1) a cover pad 264, (2) a stem 268 affixed to the cover pad 264 and extending downwards from the bottom side of the cover pad 264, and (3) a bandage 285 coupled to the stem 268.

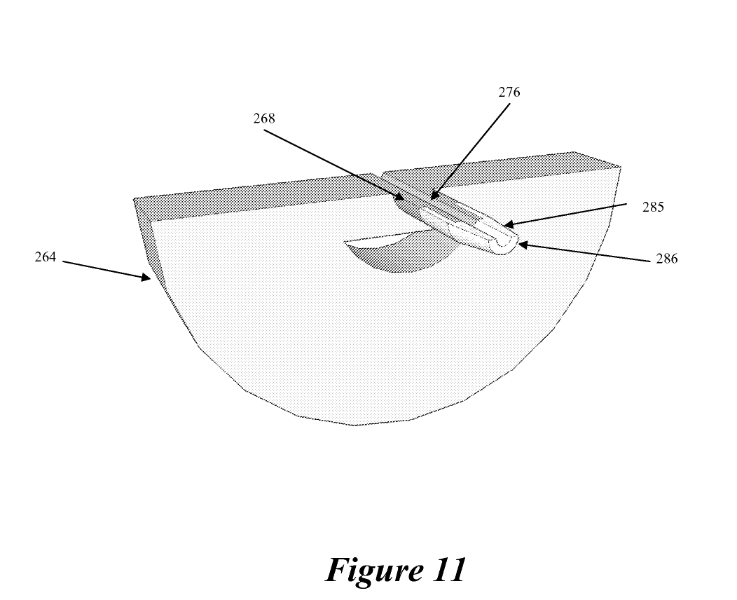

Like the bandage 85 of apparatus 60, the bandage 285 of apparatus 260 has a tapered tip. The bandage 285 or its tapered tip can be coated with, contain, or be completely composed of Chitosan or other pro-coagulant materials or agents in some embodiments. Section VI further elaborates on the structure and composition of the bandage. Like the apparatus 60, the apparatus 260 also has a central lumen 276 defined from the top of the cover pad downwards through the center of the stem 268 and through the center of the bandage 285, as illustrated in FIG. 11.

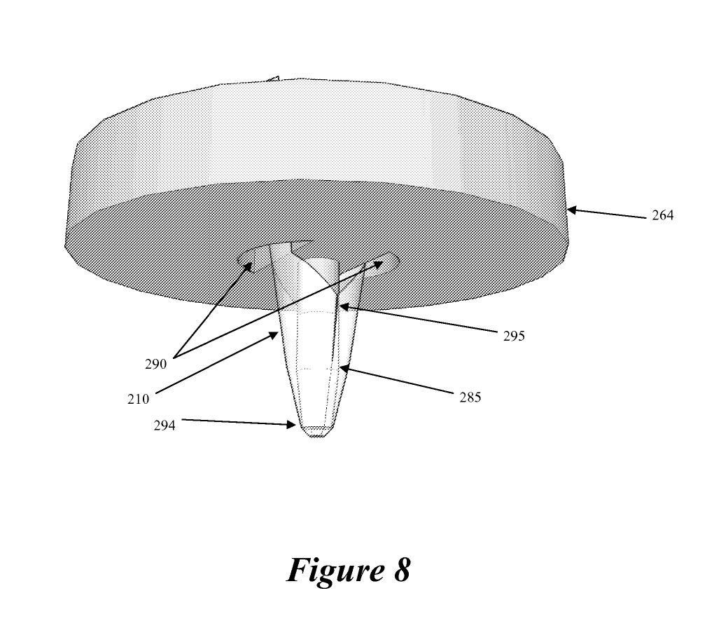

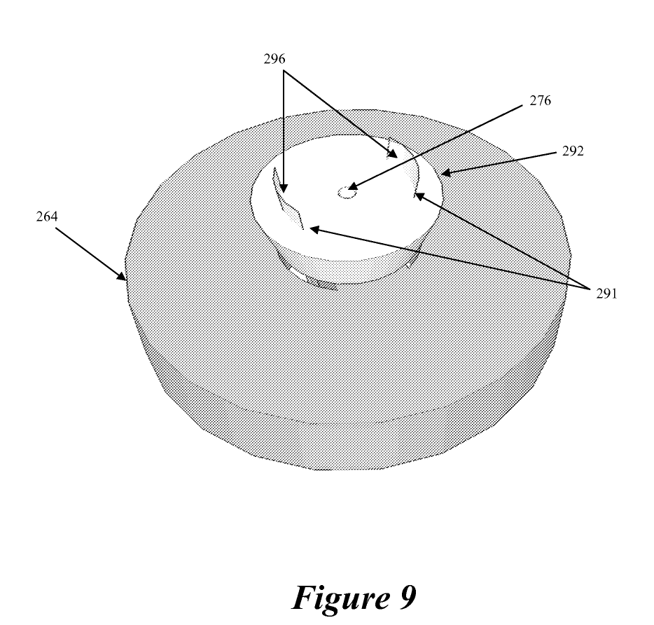

In addition to the components mentioned above, the apparatus 260 includes (1) two slots 290 in the cover pad 264, (2) a sheath 210, and (3) a holding pad 292 with two slots 291, as shown in FIGS. 8-9. The sheath 210 is coated with, or is composed partially or entirely of, a lubricious material, such as a hydrophilic polymeric film or other hydrophilic material. The lubricious sheath 210 has a hollow tip 294 that surrounds the bandage 285 (i.e., surrounds the tip). The sheath also has two flaring fingers 296 that extend from the hollow tip 294. As illustrated in FIGS. 7-9, these two fingers 296 pass through the two slots 290 of the cover pad 264 and the two slots 291 of the holding pad 292, which rests on top of the cover pad 264. In this manner, the two fingers 296 maintain the lubricious sheath's hollow tip around the bandage 285. The central lumen 276 of the apparatus 260 is defined through the top of the holding pad 292 through the center of the cover pad 264 and bandage 285.

The apparatus 260 of FIGS. 7-9 operates in a similar manner to the apparatus 60 of FIGS. 3-5. Specifically, the stem 268 and the bandage 285 of the apparatus 260 can be pushed into a puncture tract by passing the central lumen 276 of the apparatus 260 over a guidewire that is positioned in the access tract. Different embodiments deliver the hemostatic bandage 285 to different depths within the puncture tract. Section V elaborates on how different embodiments of the invention deliver the hemostatic bandage to different depths within or through the skin.

The lubricious sheath 210 assists in positioning the bandage 285 into the puncture tract. Without this sheath 210, a surgical-team member might have a hard time inserting the bandage 285 into the puncture tract. However, with the sheath, the bandage 285 can be inserted much easier into the tract, as the lubricious sheath becomes slick when it contacts liquids in the tract.

Once the bandage 285 has been placed in its desired position within the tract, the operator removes the sheath 210 from the puncture tract. The sheath 210 has slits 295 on the exterior surface of its hollow tip 294. To remove the sheath 210 from the puncture tract, the operator pulls the holding pad 292 away from the cover pad 264. This pulling causes the hollow tip 294 to tear typically along the position of the slits 295. This tearing breaks the enclosure of the shealth's tip 294 around the bandage 285, and thereby allows the sheath 210 to slide out of the puncture tract.

With the sheath 210 removed, the pro-coagulant materials or agents of the bandage 285 secure hemostasis in a few minutes. Specifically, as mentioned above, the bandage 285 can be coated with, contain, or be completely composed of Chitosan or other pro-coagulant materials or agents in some embodiments. The use of coagulating materials or agents in the bandage 285 impedes the blood flow almost immediately after the removal of the sheath 210. In addition, before or after the removal of the sheath, the guidewire is removed, and this removal causes the access tract to collapse. Also, the tissue exerts force on the tapered tip of the bandage 285 to close the hole at this tip. Accordingly, the insertion of the bandage 285, the use of the coagulating materials or agents, and the collapse of the tissue restrict the flow of blood from the blood vessel, thereby quickly and efficiently resulting in hemostasis.

To achieve hemostasis, a physician might also exert minimal pressure on the cover pad in some cases for a small duration of time (e.g., thirty to sixty seconds). Also, the bandage 285 has to remain in the patient for a suitable amount of time to secure hemostasis. This amount can be as little as 2 to 60 minutes in some cases. After a suitable period to allow for recovery and healing, the bandage 285 is removed from the living organism by pulling the cover pad 264 away from the patient. After the removal of apparatus 260, a light topical dressing might be applied to the wound.

### V. DELIVERING THE BANDAGE TO DIFFERENT DEPTHS WITHIN OR THROUGH THE SKIN

As mentioned above, different embodiments of the invention deliver the hemostatic bandage to different depths within or through the skin. Before discussing these varying depths, it is helpful to understand the relevant terminology concerning the various layers of skin. Therefore, Section A provides a background discussion of the different layers of skin in a human. Section B then provides different examples for deploying the bandage at different depths within or through the skin.

A. The Skin

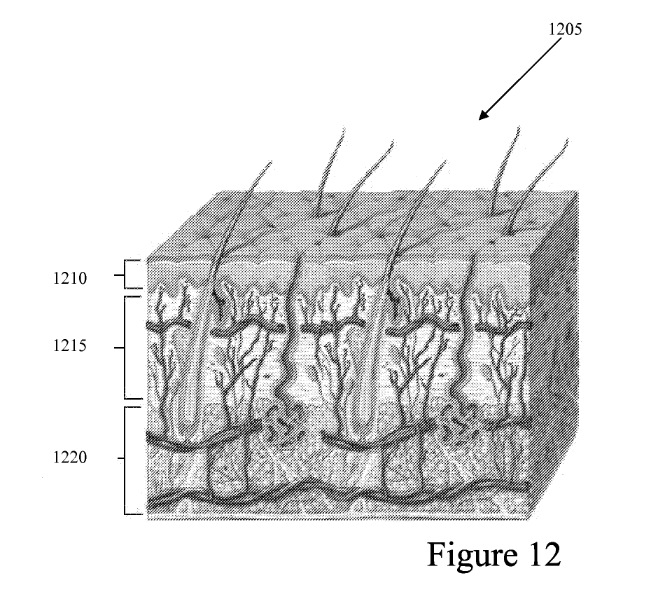

FIG. 12 provides a cross-sectional view of a person's skin 1205. As shown in this figure, the skin 1205 can be described in terms of three layers. These three layers are (1) an epidermis layer 1210, (2) a dermis layer 1215, and (3) a subcutaneous tissue layer 1220.

The epidermis layer 1210 is the external layer of skin 1205 that faces the outside world. The epidermis layer 1210 is mainly composed of cells, where each cell type serves a specific barrier function. As a result, the epidermis layer 1210 serves as a protective barrier against the external environment. The epidermis layer 1210 can be further divided into sublayers of stratum basale, stratum spinosum, stratum granulosum, stratum licidum, and stratum corneum (not shown in the figures).

The dermis layer 1215 is an internal layer coupled to the epidermis layer 1210 by a dermal-epidermal junction (not shown in figures). The dermal-epidermal junction is a structure that connects together the epidermis 1210 and dermis 1215 layers. The function of the dermis layer 1215 is to provide support and durability to nerves, vasculature, and other structures. The dermis layer 1215 resists deformation from outside forces, by returning the skin 1205 to its resting state after receiving the outside forces. The dermis layer 1215 can include collagen, elastic tissue, and reticular fibers (not shown in figures).

The subcutaneous tissue layer 1220 is located below the dermis layer 1215. This layer is important for regulating body and skin temperature. The size of this layer varies throughout the body and varies from person to person. The subcutaneous tissue layer 1220 includes a layer of fat and connective tissue that houses larger blood vessels and nerves. The third layer is sometimes referred to as hypodermis tissue.

B. Examples of Different Deployment Depths within or Through the Skin

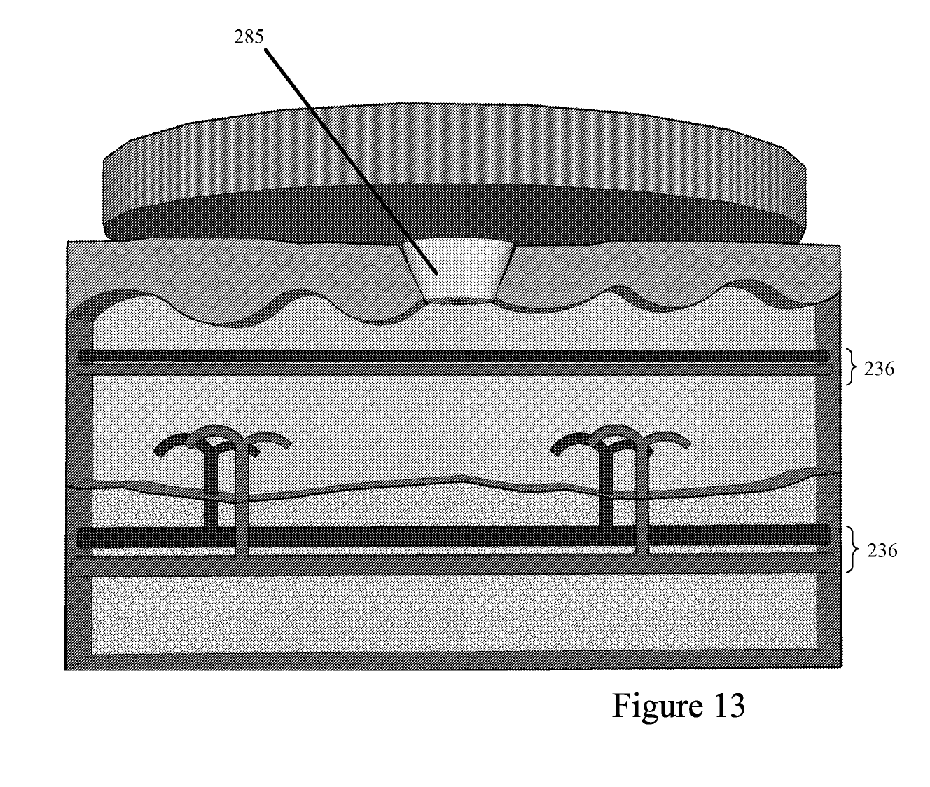

The hemostatic bandage of different embodiments (e.g., hemostatic bandage 285) can be inserted into different depths within or through the skin 1205. FIGS. 13-15 show examples of the bandage 285 inserted at different depths.

FIG. 13 shows the bandage 285 inserted into the epidermis layer 1210 of the skin 1205. As mentioned above, the epidermis layer 1210 is the external layer of skin 1205 that faces the outside world. An advantage of delivering a bandage 285 at this depth is that in some embodiments of very superficial wounds with sensitive bleeding vessels primarily just below the epidermis of the skin, the apparatus may deliver its hemostatic bandage just proximal to the sensitive bleeding sources order to exert maximal hemostatic effect without entering into sensitive bleeding structures.

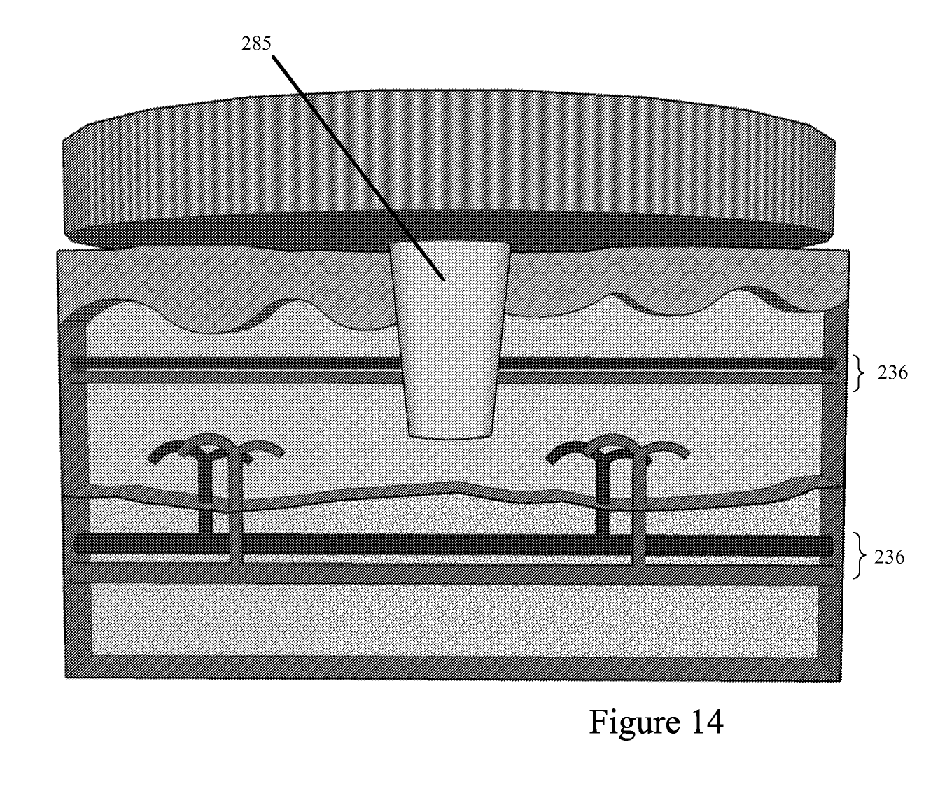

FIG. 14 shows the bandage 285 inserted into the dermis layer 1215. As mentioned above, the dermis layer 1215 is an internal layer of skin 1205 that is coupled to the epidermis layer 1210 by the dermal-epidermal junction. An advantage of delivering a bandage 285 at this depth is that in some embodiments of superficial wounds with bleeding primarily within the dermis 1215 of the skin, the apparatus may deliver its hemostatic bandage 285 directly to the bleeding sources in the dermis 1215.

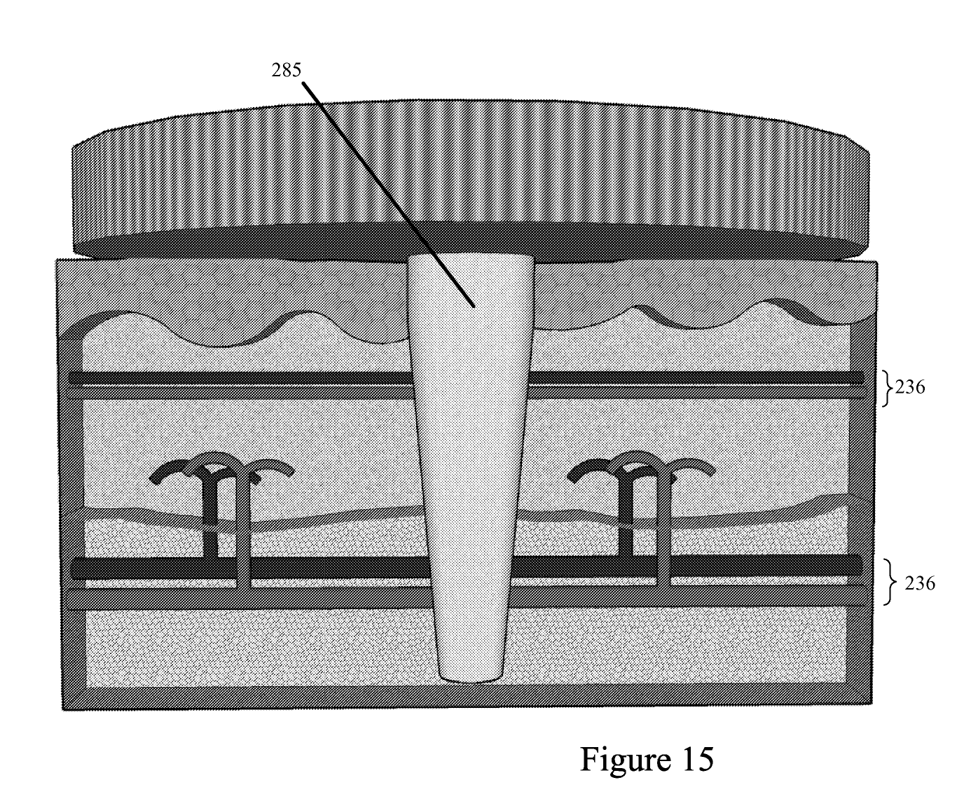

FIG. 15 shows the bandage 285 inserted into the subcutaneous tissue layer 1220. As mentioned above, the subcutaneous tissue layer 1220 is located below the dermis layer 1215. An advantage of delivering a bandage 285 at this depth is that by delivering the active bandage component into the subcutaneous tissue, the bandage can exert its hemostatic action directly on the bleeding source in some embodiments.

In FIGS. 13-15, the blood vessels are dermal and subcutaneous capillaries 236 (i.e., not the femoral or other artery). Also, the capillaries 236 (i.e., blood vessels) are not penetrated by the hemostatic bandage. In FIGS. 14-15, where the bandage 285 goes past capillaries 236, the bandage 285 is going around (i.e., not entering or going through) the capillaries 236.

In some embodiments, the bandage 285 may be removed from the living organism once adequate hemostasis has been achieved. In this manner, the bandage 285 acts as a disposable hemostatic bandage.

### VI. THE STRUCTURE AND COMPOSITION OF THE BANDAGE

As mentioned above, the structure and composition of the hemostatic bandage can be different in different embodiments. For instance, the hemostatic bandage can have different shapes in different embodiments. The bandage can also be composed of different materials in different embodiments.

Section A below describes different structures for the hemostatic bandage. Section B then describes different materials that can be used to make the bandage. Section C describes the use of different coagulating materials and agents for making or coating the bandage.

A. The Structure of the Bandage

The hemostatic bandage of some embodiments (e.g., the hemostatic bandage 285) can have different shapes in different embodiments. For instance, the hemostatic bandage can be conical, cylindrical, approximately conical/cylindrical, or any other shape. In some embodiments, the hemostatic bandage has a tapered tip.

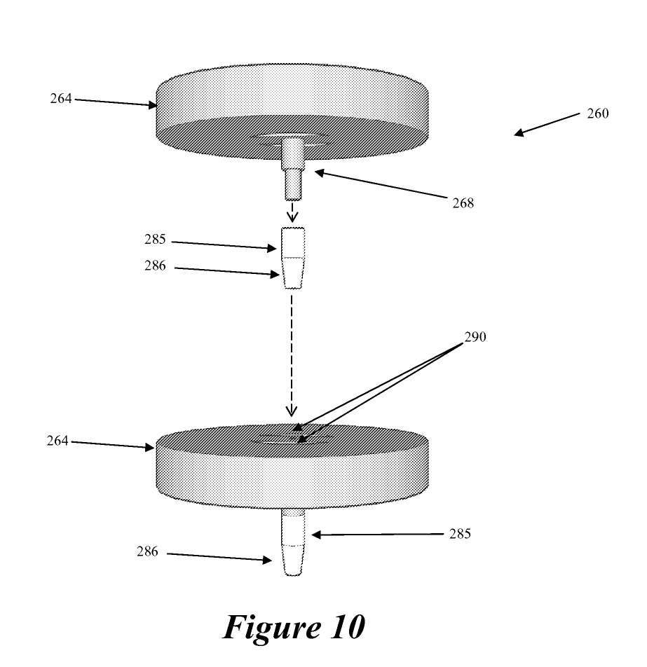

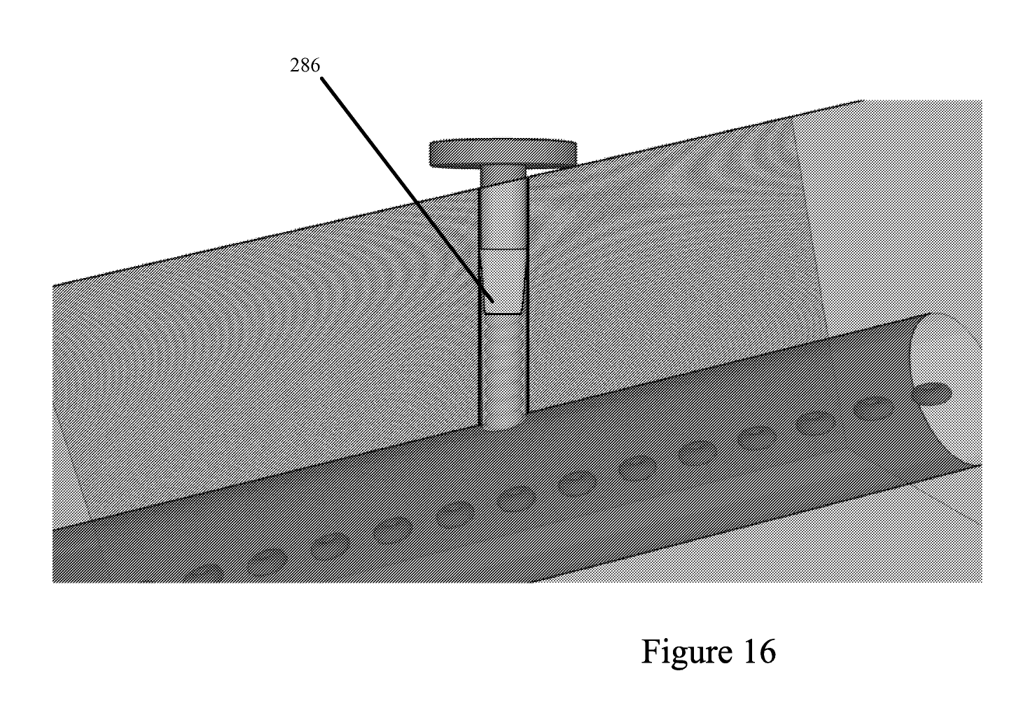

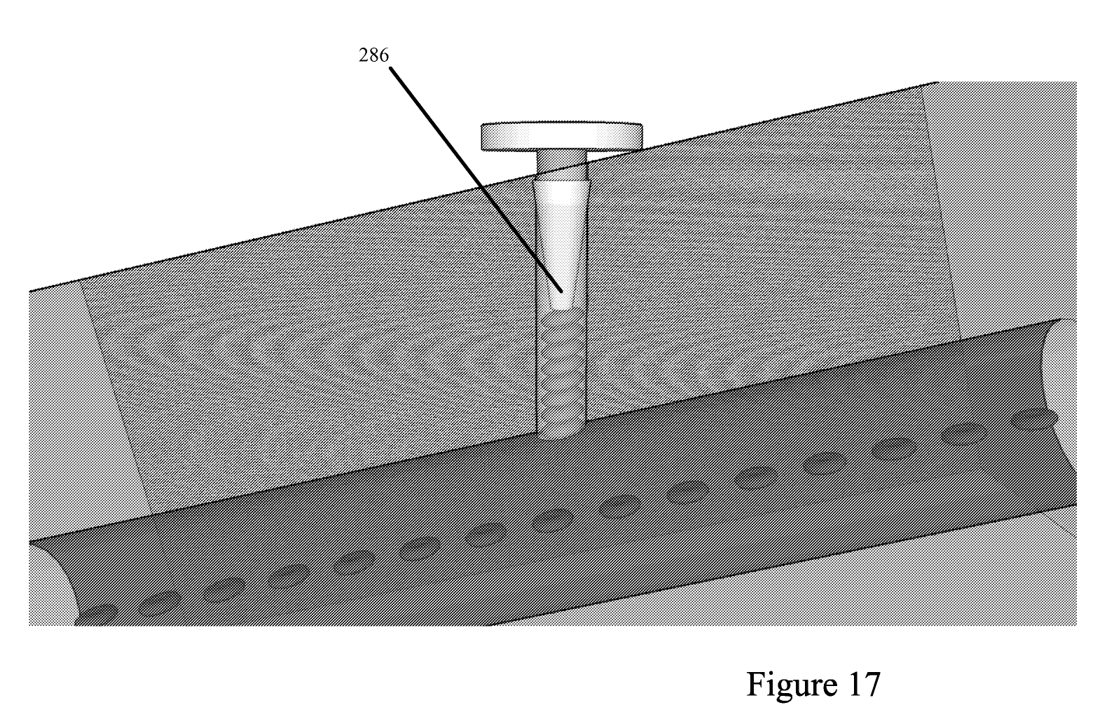

As mentioned above, the tapered tip can be a mild or sharp taper. FIGS. 10-11 and 16 show examples of a mild tapered tip 286. In some embodiments the shape of mild tapered tip is cylinderical, bullet shaped, or a blunt rounded tip. FIG. 17 shows an example of a sharp tapered tip 286. In other embodiments, the tapered tip includes any other shape. A tapered tip is especially useful when a guide wire is not used for insertion.

In some embodiments, the hemostatic bandage has a hollow interior that allows the bandage to couple onto a stem (e.g., stem 268). This coupling can be strengthened by an adhesive such as glue. In other embodiments, the hemostatic bandage and stem are not separate components. Instead, the bandage and the stem form an inseparable member/element of the hemostatic apparatus.

FIGS. 18, 19 and 20 show a bandage (e.g., bandage 385) of some embodiments that covers a majority or the entirety of its associated stem. FIGS. 18-19 show side views of an apparatus 360 with a stem 368, cover pad 364, bandage 385 and a trough 366. The trough 366 can be located in the cover pad 364 where the cover pad 364 meets the proximal end of the stem 368. The trough provides an opening for the bandage 385 to couple to or go through. The coupling of the bandage 385 at the trough 366 can be strengthened by using an adhesive material (e.g., glue) inside the trough 366.

FIG. 20 shows a top perspective view of the apparatus 360 with a center lumen 376 for an optional wire guided placement. Like the central lumen 76, the central lumen 376 the central lumen 376 is defined as an opening from the outer surface of the center of the cover pad 64, through the center of the stem 68, and through the center of the tip 386 of the bandage 385. The lumen 376 allows observation of ongoing bleeding within a vascular access or puncture tract 48. In some embodiments, a valve located on the cover pad 364 prevents back bleeding through the central lumen 376 when the valve is closed.

The central lumen 376 also gives the physician the choice to use a guide wire to facilitate insertion. The lumen 376 can act as a guide to insert the bandage 385 into a vascular puncture tract by passing a wire through the bandage 385 and the delivery mechanism.

In some embodiments, the bandage is the stem itself (i.e., the stem and the bandage are one component). In some embodiments this stem/bandage has many of the attributes of the bandage described above and below in Sections III-VI. For instance, it can be made of the same material (e.g., Chitosan, Fibrinogen, etc.), it can have a tapered tip, etc.

B. Composition

Different materials can be used to make the bandage. In some embodiments, the hemostatic bandage is made of a solid flexible material that allows the bandage to bend when the bandage is inserted into a puncture tract. In some embodiments, the bandage is composed of a solid flexible material (e.g., foam). This flexibility reduces the patient's discomfort.

In some embodiments, the hemostatic bandage is made from one or more absorbent materials. For instance, the hemostatic bandage can be composed of absorbent materials such as polymeric foam, polyurethane, hydrophilic polyurethane, etc. In some embodiments, polymeric foam is a hydrophilic polyurethane foam.

In some embodiments, the hemostatic bandage may be composed of absorbent cotton, cotton wool, or cotton gauze. In other embodiments the hemostatic bandage may be composed entirely of Chitosan. In some embodiments, the Chitosan can be a lyophilized solution molded (i.e. shaped) to the bandage configuration.

C. Coagulating Materials

In some embodiments, the hemostatic bandage may be made from, contain, or be coated with one or more pro-coagulating (i.e., coagulating) materials (i.e. coagulating agents). Coagulating materials facilitate coagulation and hemostasis. Examples of coagulating materials that can be used include Chitosan, Fibrinogen, Thrombin, self-assembling peptides, and other types of coagulating materials.

In some embodiments, the bandage 85 is coated with, contains, or is completely composed of Chitosan or other pro-coagulant (i.e., coagulant) material (i.e. coagulating agents). The use of coagulating agents in the bandage 85 further impedes the blood flow. Section VI further elaborates on the structure and composition of the bandage of some embodiments.

Coagulating materials can also include solid materials such as polyurethane and hydrophilic polyurethane. Some embodiments combine one or more coagulating materials with one or more absorbent materials to form their respective hemostatic bandage. Other embodiments coat the absorbent material of a hemostatic bandage with coagulating materials.

For example, Chitosan or an acetic solution can be incorporated on or into a foam bandage by means of dipping or soaking the bandage. In some embodiments, the foam bandage is lyophilized (e.g., cooled so that the coagulating agents crystallize or otherwise solidify) after dipping the bandage into the solution.

The use of coagulating agents in the bandage causes coagulation in the puncture tract, which further impedes the blood flow. As a result, hemostasis is achieved earlier than otherwise possible. By varying the composition of the bandage, the hemostasis rate may be controlled or varied to best fit the needs of each individual patient.

The delivery apparatus and bandage of some embodiments constitute a significant advance in the fields of cardiology, radiology and vascular surgery as it significantly improves upon the art by providing an effective means of completely sealing a vascular access puncture site, even in anti-coagulated patients, without bleeding and hematoma formation. Compared with the topical application of a bandage as used in the prior art without the precise guide wire directed positioning of the invention's insertion bandage tip, the probability of hematoma formation and the need for prolonged application of external pressure is greatly reduced by using the apparatus. The apparatus will reduce patient discomfort, improve sheath related complication rates due to bleeding and hematoma formation, eliminate intra-arterial trauma, reduce hospitalization time and allow rapid mobilization and earlier discharge of patients following catheter based vascular procedures.

While the invention has been described herein with reference to numerous specific details, one of ordinary skill in the art will recognize that the invention can be embodied in forms without departing from the spirit of the invention. For instance, the examples above describe a wire guided implementation. In other embodiments, the apparatus may be inserted without a guide wire. Several such embodiments are described in U.S. Patent Provisional Application 60/863,565, which is incorporated herein by reference.

In some embodiments, the bandage (i.e., plug) is part of an adjustable strap that is used to secure the bandage within the puncture tract and maintain pressure until hemostasis has been achieved. For instance, some embodiments are designed for a radial artery application. In some of these embodiments, the adjustable strap is an adhesive wristband that is wrapped around the patient's wrist, after the completion of an operation that uses the radial artery (which is an artery that passes through the patient's wrist). Other embodiments might implement the hemostatic device differently so that it can affix to other parts (e.g., the leg, stomach, etc.) of the patient's body. Several such embodiments are described in U.S. Patent Provisional Application 60/827,055, which is incorporated herein by reference.

Some embodiments are described above with reference to a human patient. However, as mentioned above, the invention can be used in any puncture tract for other living organisms. Thus, one of ordinary skill in the art would understand that the invention is not to be limited by the illustrative details contained herein, but rather is to be defined by the appended claims.

We claim:

1. An apparatus for achieving hemostasis, the apparatus comprising:
(a) an exterior member; and
(b) a protruding member that protrudes from the exterior member, the protruding member for inserting into a puncture tract.

2. The apparatus of claim 1, wherein the protruding member comprises a stem and a bandage, said bandage for insertion at least partially into the puncture tract.

3. The apparatus of claim 2, wherein the bandage affixes to the stem.

4. The apparatus of claim 2, wherein the bandage surrounds the stem.

5. The apparatus of claim 2, wherein the bandage and stem both connect to the exterior member.

6. The apparatus of claim 2, wherein the bandage covers a majority of the stem.

7. The apparatus of claim 2 further comprising a trough in the exterior member for coupling the bandage to the exterior member.

8. The apparatus of claim 7, wherein the trough contains an adhesive material to strengthen the coupling between the bandage and the exterior member.

9. The apparatus of claim 1 further comprising a lubricious sheath placed around the protruding member to facilitate insertion of the protruding member into the puncture tract.

10. The apparatus of claim 2 further comprising a strap coupled to the bandage to affix the bandage around a patient.

11. The apparatus of claim 1 further comprising a lumen that is defined through the exterior member and the protruding member, the lumen for observing for ongoing bleeding within the puncture tract.

12. The apparatus of claim 11, wherein the lumen is available for passing a wire through the apparatus in order to guide the protruding member into the puncture tract.

13. The apparatus of claim 2, wherein the bandage is removed from the puncture tract after hemostasis has been achieved.

14. The apparatus of claim 2, wherein the bandage has a tapered tip for easy insertion into the puncture tract without the need for a guide wire.

15. The apparatus of claim 2, wherein the bandage is shaped for easy insertion into the puncture tract.

16. The apparatus of claim 1, wherein the protruding member is delivered to epidermis layer of the skin.

17. The apparatus of claim 1, wherein the protruding member is delivered to dermis layer of the skin.

18. The apparatus of claim 1, wherein the protruding member is delivered to subcutaneous tissue layer of the skin.

19. The apparatus of claim 2, wherein the bandage comprises a solid flexible material for inserting into a puncture tract.

20. The apparatus of claim 2, wherein the bandage is made from an absorbent material.

21. The apparatus of claim 20, wherein the absorbent material is polyurethane.

22. The apparatus of claim 20, wherein the absorbent material is hydrophilic polyurethane.

23. The apparatus of claim 20, wherein the absorbent material is polymeric foam.

24. The apparatus of claim 2, wherein the bandage comprises at least one coagulating material.

25. The apparatus of claim 24, wherein the coagulating material is Chitosan.

26. The apparatus of claim 24, wherein the coagulating material is Thrombin.

27. The apparatus of claim 24, wherein the coagulating material is Fibrinogen.

28. The apparatus of claim 24, wherein the coagulating material is self-assembling peptides.

29. The apparatus of claim 2, wherein the bandage is loaded with Chitosan by dipping or soaking of the bandage in a Chitosan/acetic acid solution followed by lyophilization of the treated bandage.

30. The apparatus of claim 2, wherein the bandage is coated with Chitosan.

31. The apparatus of claim 2, wherein the bandage comprises at least one section that is entirely composed of Chitosan.

32. The apparatus of claim 1, wherein the exterior member is a cover pad for occluding the puncture tract and maneuvering the apparatus.

33. The apparatus of claim 32 further comprising an adhesive material on the cover pad for affixing the cover pad to a patient while the protruding member is within the puncture tract.

34. The apparatus of claim 32, wherein the cover pad provides a surface for a person to apply pressure to maintain the protruding member within the puncture tract.

35. The apparatus of claim 1, wherein said apparatus is a hemostatic bandage.

36. The apparatus of claim 1, wherein the protruding member comprises a bandage.

37. A method for achieving hemostasis in a puncture tract by using a device comprising a protruding member, the method comprising:
(a) inserting the protruding member into the puncture tract; and
(b) maintaining the protruding member in the puncture tract until hemostasis is achieved.

38. The method of claim 37, wherein the protruding member comprises a stem and a bandage, said bandage for insertion at least partially into the puncture tract.

39. The method of claim 38, wherein the bandage affixes to the stem.

40. The method of claim 38, wherein the bandage surrounds the stem.

41. The method of claim 38, wherein the bandage and stem both connect to an exterior member of the device.

42. The method of claim 38, wherein the bandage is removed from the puncture tract after hemostasis has been achieved.

43. The method of claim 38, wherein the bandage has a tapered tip for easy insertion into the puncture tract without the need for a guide wire.

44. The method of claim 38, wherein the bandage is shaped for easy insertion into the puncture tract.

45. The method of claim 37, wherein the protruding member is delivered to epidermis layer of the skin.

46. The method of claim 37, wherein the protruding member is delivered to dermis layer of the skin.

47. The method of claim 37, wherein the protruding member is delivered to subcutaneous tissue layer of the skin.

48. The method of claim 38, wherein the bandage is made from an absorbent material.

49. The method of claim 38, wherein the bandage comprises at least one coagulating material.

50. The method of claim 49, wherein the coagulating material is Chitosan.

51. The method of claim 37, wherein the protruding member comprises a bandage.
